(19) United States
(12) Patent Application Publication
(10) Pub. No.: US 2017/0035783 A1
(43) Pub. Date: Feb. 9, 2017

(54) COMPOSITION FOR THE PREVENTION AND THERAPY OF TUMOR DISEASES

(71) Applicant: SANOXSYS GMBH, München (DE)

(72) Inventor: Gerhard SCHALLER, München (DE)

(73) Assignee: SANOXSYS GMBH, München (DE)

(21) Appl. No.: 15/304,420

(22) PCT Filed: Apr. 15, 2015
(86) PCT No.: PCT/DE2015/000182

§ 371 Date: Oct. 14, 2016

(30) Foreign Application Priority Data
Apr. 15, 2014 (DE) 10 2014 005 513.2

(51) Int. Cl. A61K 31/57; A61K 9/00; A61K 31/4196; A61K 9/06; A61K 31/565; A61K 31/568

(52) U.S. Cl. CPC A61K 31/57; A61K 31/565; A61K 31/568; A61K 31/4196; A61K 9/06; A61K 9/0014; A61K 9/0053

(57) ABSTRACT

The invention relates to a means of prevention and therapy of hormone-sensitive tumour tissue, especially for the prevention and/or inhibition of mammary carcinoma or prostate carcinoma. The composition according to the invention is characterised by comprising steroid hormones and aromatase inhibitors. This is preferably a triple combination of the steroid hormones oestriol (E3), progesterone and testosterone and an aromatase inhibitor. Another object of the invention is the use of the composition according to the invention for the manufacture of a drug for the prevention and therapy of hormone-sensitive tumours, preferably the mammary carcinoma or the prostate carcinoma. Areas of application of the invention are the pharmaceutical industry and the life sciences, especially medicine and medical technology,

The invention relates to a composition for the prevention and therapy of hormone-sensitive tumour tissue, in particular for the prevention and/or inhibition of mammary carcinoma or prostate carcinoma. The areas of application of the invention are the pharmaceutical industry and the life sciences, in particular medicine and medical technology.

### STATE OF THE ART

When treating a variety of carcinomas, the drug intervention into the hormonal balance often plays an important role. An outstanding example of this is the mammary carcinoma. In fact, this always involves an antihormonal therapy. The interaction of the two proliferation-enhancing steroid hormones oestradiol (E2) or oestrone (E1) with the oestrogen receptors is impeded. This can be done in two different ways. Using the selective oestrogen receptor modulators of (SERM), such as tamoxifen, the oestrogen is competitively displaced from the receptor or the synthesis of the oestrogens is disrupted almost completely through the inhibition of the aromatase. The steroid metabolism is shown schematically in FIG. 1 as an overview.

In contrast, the administration of hormones as a treatment for cancer has almost no significance. Nevertheless, the proliferation (tumour growth) cannot only be stimulated, but also inhibited in vitro and in vivo with the help of various steroid hormones.

The following preparations and hormones play an important role in the organism during tumour treatment.

Aromatase Inhibitors

Aromatase is an enzyme which catalyses the decisive step of conversion of the androgens testosterone and androstenedione into oestradiol (E2) and oestrone (E1) (FIG. 1). The aromatase inhibitors exemestane, anastrozole and letrozole have been approved for the treatment of oestrogen-receptor-expressing mammary carcinoma. Their administration prevents the formation of oestrogens from androgenic precursors to the adrenal gland or the adipose tissue in the postmenopausal phase. The aromatase inhibitors lead to an inhibition of the growth of breast cancer through the massive oestrogen withdrawal (E2 and E1) (Howell et al, 2005).

Oestriol (E3)

The oestrogen receptor-11 is a potent tumour suppressor and plays a crucial role in many types of cancer, such as prostate carcinoma (Stettner et al., 2007). The ER-11 is predominantly activated by the oestriol (Zhu et al., 2006), but also by phytoestrogens such as genistein or milk thistle. Japanese women activate the ER-11 all their life, since they absorb genistein in the form of fermented soy in their diet on a daily basis. Accordingly, Japanese women have the lowest breast cancer rates in the world, just as Japanese men have the lowest prostate cancer rates (Wu et al., 2002) (FIG. 2).

Oestriol is produced in significant amounts only during pregnancy. The oestrogenic precursors from the child's liver and the synthetic capacity of the placenta are necessary for the synthesis. Outside of pregnancy only traces of the unconjugated hormone of less than 50 pg/ml can be proven. During pregnancy, the concentration rises by a factor of 400,000 to 210 ng/ml of total oestriol.

Progesterone

The corpus luteum hormone progesterone induces the formation of the progesterone receptor which consists of two isoforms A and B (Kastner et al., 1990). The activation of the isoform A causes a proliferation inhibition, while the isoform B causes an increase in proliferation which is similar to that of oestradiol. The data from large-scale observational studies show that the natural progesterone significantly reduces breast cancer risk (Lambrinoudaki, 2014).

Testosterone

Between 2001 and 2004 Glaser et al. (2013) examined the saliva of breast cancer patients (n=357) and patients with benign breast diseases (n=184) for levels of free (active) E1, E2, E3, testosterone, DHEAS, progesterone and cortisol.

The testosterone (p<0.001) and DHEAS (p<0.007) levels in breast cancer patients were significantly lower than during the controls. The E3 levels (p<0.01) were also significantly lower in the same way in breast cancer patients.

In contrast, the levels of the E1 (p<0.006) and E2 (p<0.005) were significantly increased.

No difference could be found for progesterone and cortisol. In general, women in the postmenopausal phase have decreased testosterone and DHEAS levels compared to women in sexual maturity. This applies especially to postmenopausal breast cancer patients. In contrast, the E2 levels were significantly increased in postmenopausal breast cancer patients. The E1 levels were increased in breast cancer patients, both pre- and postmenopausal (Dimitrakakis et al., 2010).

All in all, increased E1 and/or E2 levels result in an increased risk of breast cancer, while testosterone and oestriol obviously have a protective effect.

Consequently, the group of Glaser et al. (2013) developed together with a pharmacy in Cincinnati, Ohio, a subcutaneously applicable pellet which, on the one hand, contained testosterone alone and, on the other hand, a combination of testosterone and anastrozole, an aromatase inhibitor. This Dayton study comprised a total of 674 pre- and postmenopausal patients without breast cancer between 2008 and 2010. 257 received testosterone alone, while 417 women were implanted testosterone and anastrozole as a pellet. The results showed that both testosterone alone and the combination of testosterone and anastrozole could reduce the breast cancer risk in pre- and postmenopausal women (Glaser et al., 2013). Oestriol and progesterone were not taken into consideration for this treatment approach.

However, this preventive value involves side effects. The reason for this is the significant shift of oestrogen/testosterone ratio towards the testosterone. Typical side effects, as is also reported by Glaser et al., are an increase in facial hair and the appearance of acne. An increased irritability, an increase in libido and insomnia are not reported, but often described. In addition, aromatase inhibitors lead almost regularly to hot flushes, joint problems, bone pain, and general fatigue. Overall, the side effect profile is not negligible in the case of prolonged use, especially in the mentioned combination.

It remains to be seen to what extent a tumour-free status can be achieved with the combination of aromatase inhibitors and testosterone in the long run. Japanese women and men show evidence of long-term tumour suppression, achieving this long-lasting effect with the oestriol-related phytoestrogens (genistein). The demand for using oestriol in a therapeutic or preventive procedure is certainly derived from this observation. This also has the advantage that testosterone-induced side effects can be antagonised. Similarly, this also applies to the progesterone.

### GOAL AND OBJECTIVE OF THE INVENTION

The aim of the present invention is to find a new agent for the prevention and treatment of hormone-sensitive tumours, especially mammary carcinoma or prostate carcinoma, and provide it for application, which effectively controls and/or suppresses the formation and/or spread of a tumour due to its anti-proliferative effect and at the same time significantly improves the quality of life of the treated patients.

Thus the invention was based on the task of providing an individual effective composition which significantly reduces tumour growth and clearly mitigates its side effects so that it can be used for instance without hesitation preventively during the postmenopausal phase, but also in puberty together with a GnRH analogue in women.

The task of the invention is solved according to claims 1 and 9. Further possible treatment forms result from the subclaims, the description, the drawings and the examples.

### NATURE OF THE INVENTION

Initially the strong side effects such as bone and muscle aches had to be reduced using an aromatase inhibitor therapy. At first only the oestriol (E3) was used, confirmed by basic scientific findings which did not allow the expectation of a tumour-stimulating effect. Strengthened by further basic scientific findings, testosterone was additionally administered due to its libido-enhancing effect. Finally, the composition was complemented by progesterone, which is proven to inhibit tumours.

Surprisingly, this did not only result in a significant improvement in quality of life, but a significant reduction in the tumour manifestations could also be observed.

Accordingly, the invention is based on own findings that hormone-receptor-expressing tumours regress without further medication with the composition mentioned in claim 1. This is proven by the medical histories in the illustrative examples 1-3.

According to the invention a composition for therapy and prevention of tumour diseases, particularly of mammary carcinomas or prostate carcinomas is claimed which is characterised by comprising the steroid hormones oestriol, progesterone and testosterone and an aromatase inhibitor.

It could be shown that the withdrawal of proliferation-enhancing hormones surprisingly leads to a sustained containment of hormone-dependent tumour growth through the administration of an aromatase inhibitor with the simultaneous administration of anti-proliferative steroids oestriol (E3), progesterone and testosterone. The oestrogens are generally considered as proliferation-enhancing, i.e. carcinogenic. Therefore, it was not to be expected that the component oestriol (E3) would have an inhibitory effect in combination with the proliferation-inhibiting steroids progesterone and testosterone.

Furthermore such a combination also shows a significant preventive effect.

Letrozole, exemestane or anastrozole can be administered as aromatase inhibitors. The use of letrozole was found to be particularly advantageous.

The invention focuses on the effect of steroid hormones in hormone-sensitive tumour tissues. A triple combination of three anti-proliferative steroids, namely the typical pregnancy oestriol E3, the natural progesterone and testosterone (percutaneously, perorally, parenterally or as a pellet) with simultaneous administration of an aromatase inhibitor, e.g. letrozole is administered. In doing so, the formation of the proliferation-enhancing oestradiol and the oestrone from the androgenic precursors is prevented.

The administration of a triple combination of the steroid hormones oestriol (E3), progesterone and testosterone according to the invention with the simultaneous administration of an aromatase inhibitor could clearly intensify the proliferation inhibition. At the same time the administration of the composition according to the invention leads to an equally significant improvement in quality of life. The unwanted side effects of testosterone (e.g. hair growth, increase in libido, irritability) are thus considerably mitigated by the other components.

The application of the invention consists of a combined procedure:

At the beginning the proof of the expression of the hormone receptors ERa, ER, PR A+B, TR is performed in the tumour tissue.

According to the expression of the receptors the administration of the individual hormone combination is performed in accordance with the invention.

The proportions of the hormones oestriol, progesterone and testosterone in the combination may vary within a wide range. They can be contained in equal parts (1:1:1). However, progesterone is more effective as a main component in the combination and testosterone in a lesser proportion, approximately in the ratio of 3:1. Oestriol is effective in a significantly smaller amount, about one-tenth, so that a ratio of 1:20-40:5-15, preferable 1:30:10, is a preferred composition.

It should be stressed at this point that the aim of the application is an individual hormone combination which results from the expression of the receptors and other parameters collected before treatment. The physical and psychological feelings such as increasing libido, irritability, increased blood pressure, etc. are decisive here and necessitate an individual adjustment.

The invention furthermore relates to the use of the composition according to the invention for the manufacture of a drug for the prevention and therapy of malignant tumours especially of mammary carcinomas or prostate carcinomas and/or for the treatment of metastases.

The use of the composition according to the invention is characterised by the composition being administered percutaneously, perorally, parenterally or as a pellet.

Moreover, the use is characterised by the steroid hormones oestriol, progesterone and testosterone being administered in the morning and in the evening as an ointment formulation for application to the skin. Preferably 2 g/100 kg bodyweight of the ointment formulation of the steroid hormones oestriol, progesterone and testosterone are massaged into the skin in the morning and in the evening.

Administering the steroid hormones oestriol, progesterone and testosterone separately from the aromatase inhibitor has been found to be advantageous. Thereby, the aromatase inhibitor is preferably administered orally.

Hereinafter, the present invention is described in detail using a concrete application example. It describes the administration as a skin cream with a recipe of:
Recipe: Oestriol 0.1 g plus progesterone 3.0 g plus testosterone 1.0 g plus ointment base ad 100 g
   plus 2.5 mg/d letrozole oral

1 g of this cream is massaged into the skin of the forearm in the morning and in the evening. In addition, an aromatase inhibitor (e.g. letrozole 2.5 mg) is administered every day. This combination with the aromatase inhibitor is mandatory in the case of simultaneously existing oestrogen receptor expressive tumour in order to avoid the immediate conversion of the supplied testosterone into the unwanted metabolites oestradiol and oestrone.

Table 1 shows preferred formulations of oestriol/progesterone/testosterone along with the daily dose to be administered in comparison with the commercial preparations Intrinsa, Tostran and Ovestin cream.

TABLE 1

| | Recipe no 1: | Recipe no 2: (1 ml | | Tostran 1 puff | Ovestin cream |
|---|---|---|---|---|---|
| | (2 g cream/d) | puff/d) | Intrinsa/d | (0.5 g/d) | (0.5 g/d) |
| Oestriol | 2 mg | 15 mg | | | 0.5 mg |
| Progesterone | 60 mg | 45 mg | | | |
| Testosterone | 20 mg | 1.5 mg | 0.3 mg | 10 mg | |

For prevention, the combination of oestriol, progesterone and testosterone is administered with an aromatase inhibitor in postmenopausal women.

Advantages of the composition according to the invention for the prevention and treatment of tumour diseases through the triple medication oestriol, progesterone and testosterone plus aromatase inhibitors are the following:
Significant inhibition of the proliferation rate which goes beyond a synergistic effect and consequently an increase in the survival rates
Alleviation of the side effects of testosterone and consequently significant improvement in quality of life
Avoidance of unnecessary steroid hormone administrations
Significant reduction of the fatigue syndrome
Increase in general vitality
Significant reduction of metastasis-related pain
Regression of tumour manifestations.

Hereinafter, the present invention will be described more specifically by means of examples. However, the following examples are given by way of explanation only and consequently the present invention is not limited by them or to them.

### Illustrative Embodiments

### EXAMPLE 1

Diagnosis:

C50.2GL/mammary carcinoma of the upper inner quadrant

E05.0Z/hyperthyreosis with diffuse struma

F41.2G/anxious and depressed mood

E55.9G/vitamin D deficiency

R78.8G/proof of an abnormal blood lithium value

Histology:

Lobular mammary carcinoma G2

ER 9/12, PR 9/12, HER 2+, FISH not amplified, Ki-67 10%.

Procedure:

1 Letrozole (neoadjuvant)

2 Hormone cream (neoadjuvant oestriol, progesterone and testosterone)

3 Dekristol

4 Lithium ortotate

Case History

57-year-old patient who underwent a punch biopsy of a mammary carcinoma on the upper inner left for the first time on 22 Aug. 2013. The patient had palpated a tumour there herself one year before. The patient was presented for reasons of a neoadjuvant therapy. However, she strictly rejected both surgery and chemotherapy and radiation. In the further case history thyroid surgery in 1998 due to hyperthyroidism. Two terminations of pregnancy, menopausal phase at age 53 years, menarche at the age of 13 years. From the age of 17-30 oral ovulation inhibitors, several knee operations. Since 3 years increasing exercise-induced dyspnoea (COPD).

Case History:

Inconspicuous

Course:

The above-mentioned hormone therapy was discussed with the patient. She has been extensively informed of the experimental nature of the treatment and possible side effects. She gave her consent. On 4 Oct. 2013 we started the above-mentioned treatment. The course of treatment was followed by both mammography and ultrasound. On 4 Oct. 2013 a coarse nodular tumour with a diameter of 5 cm could be palpated at the upper inner left breast between 12 o'clock and 1 o'clock. A discrete skin retraction could be seen there. The plateau phenomenon was positive. A peau d'orange was not visible. In the course, the tumour increasingly reduced in size during treatment. On 18 Mar. 2014 a tumour with a mere size of 1-2 cm could be palpated at the upper inner quadrant. Compared to 22 Aug. 2013 the control mammography on 27 Jan. 2014 showed a regression of the tumour from 4.0 to 2.5 cm. The ultrasound of the breasts dated 18 Mar. 2014 showed a regression from 4.3 to 1.2 cm in comparison to the examination dated 27 Sep. 2013 (FIG. 1). The discretely elevated tumour marker CA 15-3 decreased in the course from 32 U/ml (26 Sep. 2013) to 30 U/ml (16 Jan. 2013). The BET was discussed again with the patient. However, she rejected further conservative treatment. At that time, there was no evidence of remote metastasis. The quality of life of the patient was excellent. We substituted a pronounced vitamin D and lithium deficiency accordingly.

FIG. 3 shows the regression of a hormone-receptor-expressing mammary carcinoma under oestriol, progesterone, testosterone and aromatase inhibitor letrozole within 6 months.

The other examples show that metastases can also be additionally regressed and the accompanying pain is stopped.

### EXAMPLE 2

Diagnosis:

Ovarian carcinoma

Bone metastases L

Endplate compression fracture T12 and L2

Histology:

Poorly differentiated adenocarcinoma emanating from the ovary

pT3c, pN1 (25/34) MX L1, V1 G3 FIGO IIIC

ER pos.

Procedure:

Carboplatin taxol—chemotherapy (15 Apr. 2010-31 Jan. 2013)

Avastin q3 (24 Oct. 2012-31 Jan. 2013)

Xgeva (11 Mar. 2013-4 Jun. 2013)

Bondronat q4 (18 Jun. 2013-continuous)

Hormone cream (oestriol, progesterone, testosterone, beginning 4 Jun. 2013-continuous)

Letrozole (beginning 25 Feb. 2014-continuous)

Dekristol 10.000 IU/d

Case History

The 67-year-old patient noticed an increase in her girth since December 2009. In magnetic resonance imaging on 26 Jan. 2010 the suspicion of ovarian cancer was mentioned. On 9 Feb. 2010 an explorative laparoscopy was performed. On 26 Feb. 2010 the laparotomy of the longitudinal section followed. Uterus and ovaries, greater omentum and retroperitoneal lymph nodes were removed.

In the further case history

Cholecystectomy, appendectomy, 1 spontaneous partition, menopausal phase at the age of 50 years

Case History:

Inconspicuous

Course:

The patient initially completed guideline-based chemotherapy which was tolerated relatively well. This was supplemented by Avastin q3 in the further course. In March 2013 strong to severe pain occurred in the lower spine. The MRI dated 6 Mar. 2013 showed old endplate impressions in L2 and 4 in the lumbar and sacral region. There was a suspicion of bone metastases. Due to increasing pain in the area of the lower spine the medication with Xgeva was replaced by Bondronat infusions in June 2013.

On 4 Jun. 2013 treatment with the above-mentioned hormone cream was started. This resulted in a reduction of the pain, however, it did not stop completely.

The MRI dated 11 Mar. 2014 showed the known old vertebral body fractures. Changes suggestive of bone metastases were no longer detectable. The tumour marker which was initially clearly increased with 82 U/ml (15 Oct. 2012) returned to a normal value with 24 U/ml (13 Aug. 2013).

On 25 Feb. 2014 letrozole was administered in addition to the hormone cream as “off-label” medication. This led to complete disappearance of the pain. Now, almost 4 years after the initial diagnosis of a widespread, prognostically very unfavourable ovarian cancer the patient feels clinically excellent during treatment with the hormone cream and the etrozole.

### EXAMPLE 3

Diagnosis:

Mammary carcinoma upper outer right

Bone metastases

Vitamin B deficiency

Histology:

Ductal mammary carcinoma

pT1 (1.3 cm), pN1 (1/9), M1 bone metastases, G2

ER 12/12, PR 9/12, HER2 negative

Procedure:

1 Letrozole

2 Hormone cream (oestriol, progesterone, testosterone)

3 Bondronat

Case History

71-year-old patient in whom mammary carcinoma on the upper outer right had been proven for the first time on 25 Aug. 2005. On the same day BAT and axillary dissection and radiation therapy of the residual breast

Initially no anti-hormone therapy due to severe side effects in the gastrointestinal area. Due to increased tumour marker (approx. 15-3 93 U/ml) on 25 Sep. 2012 execution of a bone scintigram with evidence of multiple bone metastases at the calvaria in the thoracic spine, lumbar spine and the sacral vertebrae. Otherwise no further evidence of metastases. 2012 polyp removal from the urinary bladder,

borreliosis known since 2001

2009 herpes zoster

Due to a sensitive stomach, gastroscopy and colonoscopy in 2009: inconspicuous.

Three pregnancies, 1 abortion, menarche at the age of 17 years, menopausal phase at the age of 50 years

Case History:

Maternal aunt cervical carcinoma

Course:

The patient rejected all guideline-based treatments.

In May 2013, treatment with exemestane and hormone cream (oestriol, progesterone and testosterone and Bondronat) was started due to the bone metastases. The pain at the bone desisted without painkillers. The MRI on 21 Feb. 2014 showed a clear regressive bone metastasis formation. The tumour marker CA 15-3 decreased from 560 U/ml (11 Jun. 2013) to 485 U/ml (30 Jan. 2014). The patient is currently feeling very well from a clinical point of view.

### DESCRIPTION OF THE FIGURES

FIG. 1 Schematic representation of the steroid metabolism

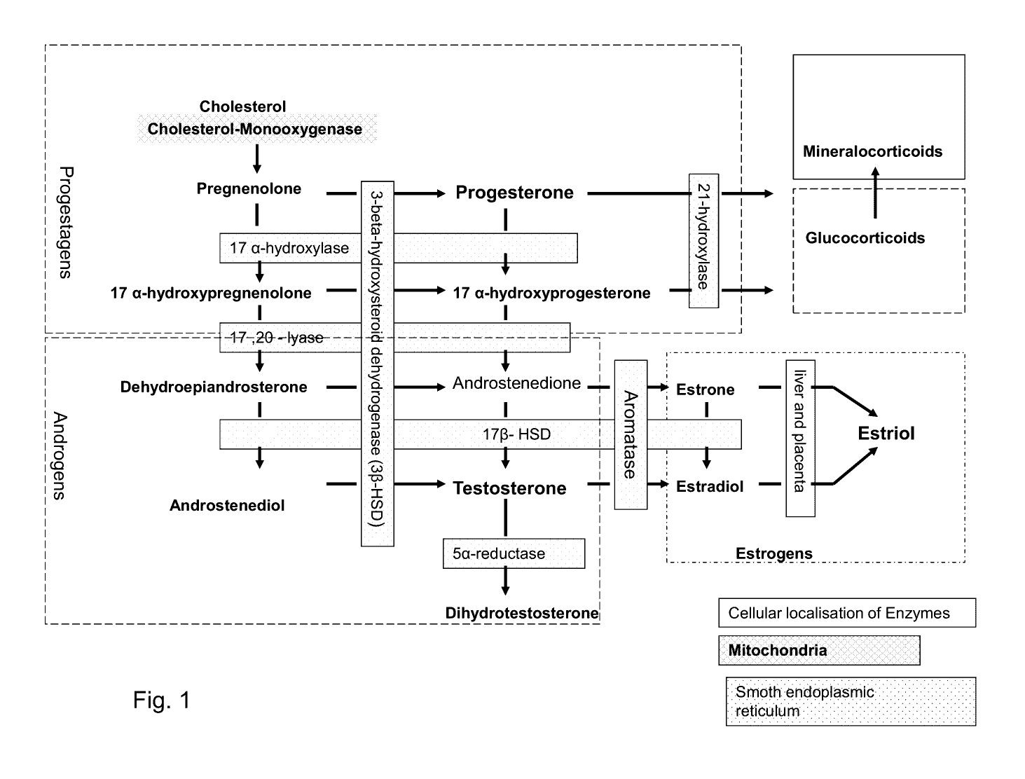

FIG. 2 Frequency of hormone-dependent cancer diseases per 100,000 per year in Northern and Southern Europe as well as East Asia where a soy-rich diet is predominant (according to IACR/WHO)

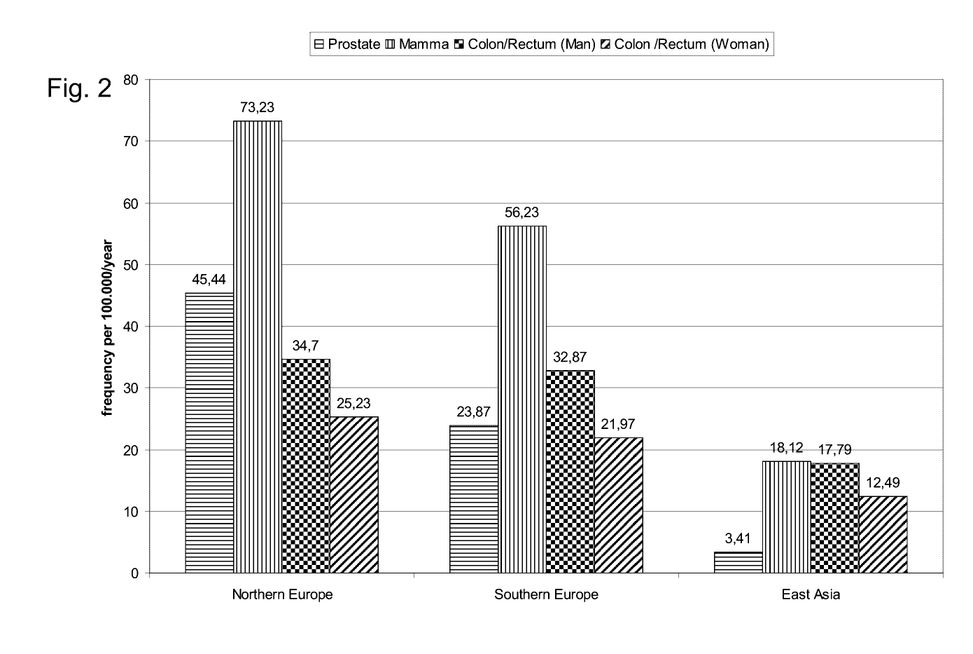

FIG. 3 Regression of a hormone-receptor-expressing mammary carcinoma within a period of almost six months under oestriol, progesterone, testosterone and aromatase inhibitor letrozole.

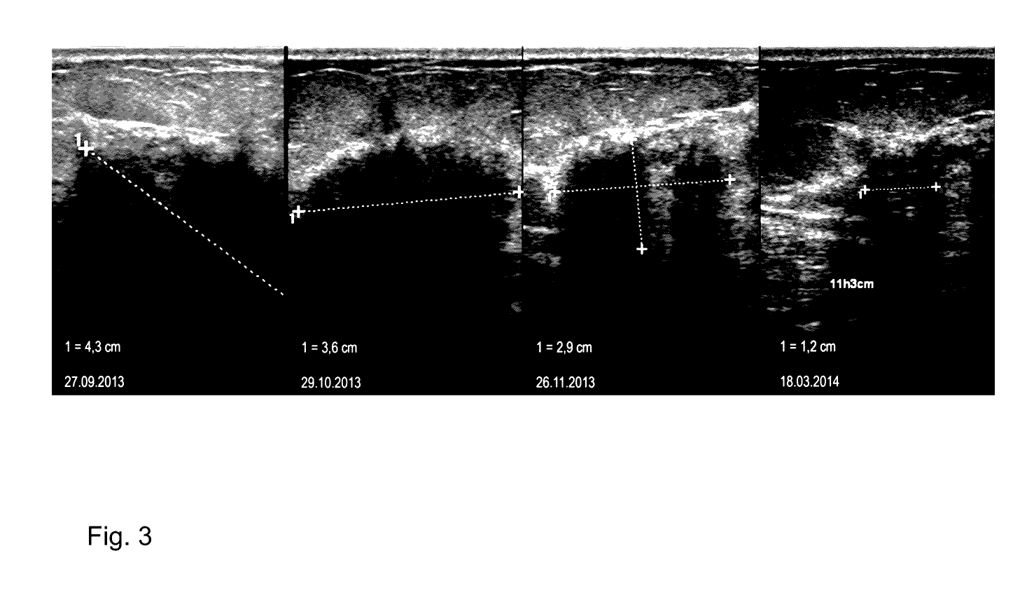

### BIBLIOGRAPHY

Howell, A; Cuzick, J; Baum, M; Buzdar, A; Dowsett, M; Forbes, J F; Hoctin-Boes, G; Houghton, J et al.: Results of the ATAC (Arimidex, Tamoxifen, Alone or in Combination) trial after completion of 5 years' adjuvant treatment for breast cancer. The Lancet 2005; 365: 60
Stettner M, Kaulfuss S, Burfeind P: The relevance of oestrogen receptor-beta expression to the antiproliferative effects observed with histone deacetylase inhibitors and phytoestrogens in prostate cancer treatment. Mol Cancer Ther. 2007; 5: 2626
Zhu B T 1, Han G Z, Shim J Y, Wen Y, Jiang X R.: Quantitative structure-activity relationship of various endogenous oestrogen metabolites for human oestrogen receptor alpha and beta subtypes: Insights into the structural determinants favouring a differential subtype binding. Endocrinology. 2006; 147:4132
Wu A H, Wan P, Nankin J, Tseng C C, Yu M C, Pike M C: Adolescent and adult soy intake and risk of breast cancer in Asian-Americans. Carcinogenesis. 2002; 23:1491
Kastner P, Krust A, Turcotte B, Stropp U, Tora L, Gronemeyer H, Chambon P (1990). “Two distinct oestrogen-regulated promoters generate transcripts encoding the two functionally different human progesterone receptor forms A and B”. EMBO J. 1990; 9: 1603
Lambrinoudaki I: Progestogens in postmenopausal hormone therapy and the risk of breast cancer. Maturitas. 2014
Dimitrakakis C, Zava D, Marinopoulos S, Tsigginou A, Antsaklis A, Glaser R.: Low salivary testosterone levels in patients with breast cancer. BMC Cancer. 2010; 10:547
Glaser R L, Dimitrakakis C. Reduced breast cancer incidence in women treated with subcutaneous testosterone, or testosterone with anastrozole: a prospective, observational study. Maturitas. 2013; 76:342

1. A composition for therapy and prevention of tumour diseases, comprising steroid hormones oestriol, progesterone and testosterone and an aromatase inhibitor.

2. The composition according to claim 1, a proportion of the steroid hormones oestriol (E3)/progesterone/testosterone being 1:20-40:5-15.

3. The composition according to claim 1, a proportion of the steroid hormones oestriol (E3)/progesterone/testosterone being 1:30:10.

4. The composition according to claim 1, wherein the composition is an ointment formulation.

5. The composition according to claim 1, the concentration of oestriol (E3) being 0.1 g/100 g, the concentration of progesterone being 3 g/100 g and the concentration of testosterone being 1 g/100 g.

6. The composition according to claim 1, wherein the aromatase inhibitor comprises a compound selected from the group consisting of letrozole, exemestane and anastrozole.

7. The composition according to claim 6, wherein the aromatase inhibitor comprises letrozole.

8. The method according to claim 16, wherein the aromatase inhibitor is letrozole and 2.5 mg/d of letrozole is administered.

9. A method for treating a patient in need of treatment for the prevention or therapy of malignant tumors, comprising administering the composition of claim 1.

10. The method according to claim 9, the composition being administered by an administration form selected from the group consisting of percutaneously, perorally, parenterally and as a pellet.

11. The method according to claim 16, the steroid hormones oestriol, progesterone and testosterone being administered in the morning and in the evening as ointment formulation.

12. The method according to claim 16, the steroid hormones oestriol, progesterone and testosterone being administered as an ointment for application to the skin.

13. The method according to claim 16, 2 g/100 kg body weight of an ointment formulation of the steroid hormones oestriol, progesterone and testosterone being massaged into the skin in the morning and in the evening.

14. The method according to claim 16, the steroid hormones oestriol, progesterone and testosterone being administered separate from the aromatase inhibitor.

15. The method according to claim 16, the aromatase inhibitor being administered orally.

16. A method for treating a patient in need of treatment for the prevention or therapy of malignant tumors, comprising administering steroid hormones oestriol, progesterone and testosterone and an aromatase inhibitor.

17. The method according to claim 13, 2.5 mg/d of letrozole being administered orally.

18. The method according to claim 13, the ointment formulation of the steroid hormones oestriol, progesterone and testosterone comprising 0.1 g/100 g of oestriol (E3), 3 g/100 g of progesterone and 1 g/100 g of testosterone.

19. The method according to claim 9, wherein the patient is in need of treatment for metastases of the malignant tumors.

20. The method according to claim 9, wherein the malignant tumors comprise malignant tumors selected from the group consisting of mammary carcinomas and prostate carcinomas.
